(19) United States
(12) Patent Application Publication
(10) Pub. No.: US 2023/0151073 A1
(43) Pub. Date: May 18, 2023

(54) FIBRILLATION RESISTANT CALCITONIN PEPTIDES AND USES THEREOF

(71) Applicant: Purdue Research Foundation, West Lafayette, IN (US)

(72) Inventors: Elizabeth M. Topp, West Lafayette, IN (US); Karthik B. Chandrababu, West Lafayette, IN (US); Harshil K. Renawala, Thana (IN)

(73) Assignee: Purdue Research Foundation, West Lafayette, IN (US)

(21) Appl. No.: 17/914,207

(22) PCT Filed: Mar. 26, 2021
(86) PCT No.: PCT/US2021/024278

§ 371 Date: Sep. 23, 2022

Related U.S. Application Data
(60) Provisional application No. 63/001,599, filed on Mar. 30, 2020.

(51) Int. Cl. C07K 14/585

(52) U.S. Cl. CPC C07K 14/585

(57) ABSTRACT

The present invention generally relates to peptides with no or substantially reduced fibrillation and their therapeutic uses, in particular to phosphorylated calcitonin peptides that have no measureable or substantially reduced fibrillation in an aqueous environment. Also described herein are pharmaceutical compositions of such compounds and methods for treating a patient suffering from pain, osteoporosis, Paget's disease, and/or hypercalcemia by administering therapeutically effective amounts of such compound alone, or together with other therapeutics, or in a pharmaceutical composition to a patient in need of relief.

### CROSS REFERENCE TO RELATED APPLICATIONS

The present U.S. patent application is related to and claims the priority benefit of U.S. Provisional Patent Application Ser. No. 63/001,599, filed Mar. 30, 2020, the contents of which are hereby incorporated by reference in their entirety into the present disclosure.

### GOVERNMENT SUPPORT CLAUSE

This invention was made with government support under GM085293 awarded by the National Institutes of Health. The government has certain rights in the invention.

### STATEMENT OF SEQUENCE LISTING

A computer-readable form (CRF) of the Sequence Listing is submitted with this application. The file, generated on Mar. 16, 2021, is entitled 68428-02_Seq_Listing_ST25_txt, the contents of which are incorporated herein in their entirety. Applicant states that the content of the computer-readable form is the same and the information recorded in computer readable form is identical to the written sequence listing.

### TECHNICAL FIELD

The present invention generally relates to peptides with no or substantially reduced fibrillation and their therapeutic uses, in particular to phosphorylated calcitonin peptides that have no measurable or substantially reduced fibrillation in an aqueous environment. Also described herein are pharmaceutical compositions of such compounds and methods for treating a patient by administering therapeutically effective amounts of such compound alone, or together with other therapeutics, or in a pharmaceutical composition.

### BACKGROUND

This section introduces aspects that may help facilitate a better understanding of the disclosure. Accordingly, these statements are to be read in this light and are not to be understood as admissions about what is or is not prior art.

Calcitonin is a 32 amino acid peptide hormone (SEQ ID NO: 1) secreted by parafollicular cells (also known as C cells) of the thyroid gland in humans, and in many other animals in the ultimopharyngeal body. It acts to reduce blood calcium (Ca²⁺), opposing the effects of parathyroid hormone (PTH). Calcitonin has been found in fish, reptiles, birds, and mammals.

In its skeleton-preserving actions, calcitonin protects against calcium loss from the skeleton during periods of calcium mobilization, such as pregnancy and, especially, lactation. The protective mechanisms include the direct inhibition of bone resorption and an indirect effect through the inhibition of the release of prolactin from the pituitary gland. Other effects are in preventing postprandial hypercalcemia resulting from absorption of Ca²⁺. Also, calcitonin inhibits food intake in rats and monkeys, and may have CNS action involving the regulation of feeding, appetite, and mood.

Calcitonin can be used therapeutically for the treatment of hypercalcemia or osteoporosis. In a recent clinical study, subcutaneous injections of calcitonin reduced the incidence of fractures and reduced the decrease in bone mass in women with type 2 diabetes complicated with osteoporosis. Subcutaneous injections of calcitonin in patients suffering from mania resulted in significant decreases in irritability, euphoria and hyperactivity and hence calcitonin holds promise for treating bipolar disorder. Widespread use of calcitonin peptide is hindered, at least partially, because the calcitonin peptide is not stable in an aqueous solution and rapidly undergoes fibrillation. The more slowly aggregating salmon calcitonin (sCT), which has 50% sequence homology to hCT, is used in clinical settings instead. However, sCT therapy has been associated with side effects such as vomiting and anorexia, and with immune responses such as resistance and antibody formation, limiting its use in certain patient populations. In addition, hCT has shown greater potency than sCT under conditions in which its fibrillation could is controlled. Thus, there is an unmet clinical need for a fibrillation-resistant hCT.

### BRIEF DESCRIPTION OF THE DRAWINGS AND SEQUENCE LISTING ID

The above and other objects, features, and advantages of the present invention will become more apparent when taken in conjunction with the following description and drawings, wherein:

FIG. 1: Fibrillation kinetics of human calcitonin (hCT) by ThioT fluorescence (solid circles) and turbidity (solid triangles) measurements. hCT was dissolved in 20 mM sodium phosphate buffer pH 7.4 at 100 μM and allowed to fibrillate at 25° C. with continuous shaking at 731 cpm in 96-well microtiter plate. n=6, mean±SD.

FIGS. 2A-2B: HDX kinetics of monomeric human calcitonin (hCT). (2A) Percent deuterium uptake for intact hCT as a function of time (0.5-5 min). (2B) Percent deuterium uptake for six peptic digest hCT fragments as a function of time (0.5-5 min). Lines represent regression to a mono-exponential association model (Eq. 2) (n=3, mean±SD). Standard deviation bars are smaller than the size of the symbols;

FIGS. 2C-2D: HDX kinetics for monomeric human calcitonin (hCT). (2C) Percent deuterium uptake for intact hCT as a function of time. (2D) Percent deuterium uptake for six peptic digest hCT fragments as a function of time. Lines represent regression to a mono-exponential association model (Eq. 2) (n=3, mean±SD). Standard deviation bars are smaller than the size of the symbols; and

FIG. 2E: HDX-MS of monomeric human calcitonin (hCT). Enhanced mass envelopes of intact hCT (m/z=1139.5416, z=+3) and deuterated hCT recorded at various times (0.5-120 min). Deuterated mass envelopes show a single Gaussian peak consistent with deuteration of monomeric peptide. #D is the average number of deuterons incorporated (n=3).

FIG. 3: Pulsed HDX-MS of fibrillating human calcitonin (hCT) at intact level. Fibrillating hCT was pulse labelled with deuterium for 2 min at intervals over the fibrillation time course (to 1440 min) and analyzed for deuterium uptake by MS. Enhanced mass envelope of deuterated and undeuterated hCT (m/z=1139.5416, z=+3) at intact level. The spectra show a bimodal peak distribution (Peaks I & II) during fibrillation. % D is the average percent deuterium uptake by hCT (n=3).

FIG. 4: Pulsed HDX-MS of fibrillating human calcitonin (hCT) at intact level. Fibrillating hCT was pulse labelled with deuterium for 2 min at intervals over the fibrillation time course (to 1440 min) and analyzed for deuterium uptake by MS. Fibrillation kinetics of hCT by ThioT fluorescence (solid circles, n=6), turbidity (solid triangles, n=6), and deuterium uptake by HDX-MS (solid squares, n=3). Lines are fits to Boltzmann sigmoid function (Eq. 1). Mean±SD.

FIG. 5 and FIG. 6: Pulsed HDX-MS of fibrillating human calcitonin (hCT) at fragment level. Fibrillating hCT was pulse labelled with deuterium for 2 min at intervals over the fibrillation time course (to 1440 min) and analyzed for deuterium uptake by MS after pepsin digestion.

FIG. 5 shows t₅₀ values for deuterium incorporation in three hCT fragments (n=3, mean±SD).

FIG. 6 shows percent deuterium uptake for hCT fragments 1-8, 1-11, 12-19, 16-28, 20-32, and 26-32, as a function of time of fibrillation. Lines represent fit to Boltzmann sigmoid function (Eq. 1) in cases where good fit was achieved (n=3, mean±SD).

FIG. 7: CD spectra of calcitonins during fibrillation (0-1440 min). Overlay of CD spectra from fibrillating human calcitonin (hCT).

FIGS. 8A-8F: Effect of site-specific phosphorylation on the fibrillation of three phosphorylated human calcitonin (phCT) derivatives: phospho-Ser-5 hCT (FIGS. 8A, 8B), phospho-Thr-13 hCT (FIGS. 8C, 8D) and phospho-Thr-21 hCT (FIGS. 8E, 8F) (100 μM peptide in 20 mM sodium phosphate buffer, pH 7.4, 25° C., continuous shaking at 731 cpm for 24 h in 96-well microtiter plate). Fibrillation was monitored by ThioT florescence (RFU) at 482 nm (FIGS. 8A, 8C, 8E) and turbidity (OD) at 340 nm (FIGS. 8B, 8D, 8F). n=6, mean±SD.

FIGS. 9A-9B: Fibrillation kinetics of human calcitonin (hCT) by ThioT fluorescence (FIG. 9A) and turbidity (FIG. 9B) measurements. hCT was dissolved in 20 mM sodium phosphate buffer pH 7.4 at a concentration of 100 μM and allowed to fibrillate at 25° C. with continuous shaking at 731 cpm in 96-well microtiter plate. n=6, mean±SD.

FIGS. 10A-10C: Stability of phospho-Thr-13 hCT under stressed conditions (100 μM peptide in 20 mM sodium phosphate buffer, pH 7.4, 25° C., shaking at 1096 cpm for 14 h). Fibrillation was monitored by measuring ThioT florescence (RFU) at 482 nm (FIG. 10A) and turbidity (OD) at 340 nm (FIG. 10B). n=3, mean±SD;

FIG. 10C: Stability of phospho-Thr-13 hCT under fibrillation conditions (100 μM peptide in 20 mM sodium phosphate buffer, pH 7.4, 25° C., continuous shaking at 731 cpm for 24 h in 96-well microtiter plate) by HDX-MS. Samples were withdrawn after 24 h and subjected to a 2 min pulsed deuteration followed by MS analysis to measure deuterium uptake. Enhanced mass envelope of intact pThr-13 hCT (m/z=1166.2050, z=+3) shows no significant change in peak position and distribution after 24 hr. #D is the average number of deuterons incorporated (n=3).

FIG. 11A: CD spectra of phospho-Thr-13 hCT under fibrillating conditions (100 μM peptide in 20 mM sodium phosphate buffer, pH 7.4, 25° C., continuous shaking at 731 cpm for 24 h in 96-well microtiter plate); FIGS. 11B-11C: CD spectra of phospho-Ser-5 hCT (FIG. 11B) and phospho-Thr-21 hCT (FIG. 11C) under fibrillation conditions (100 μM peptide in 20 mM sodium phosphate buffer, pH 7.4, 25° C., continuous shaking at 731 cpm for 24 h in 96-well microtiter plate).

FIG. 12 shows amino acid sequence of human calcitonin (hCT) (SEQ ID NO: 1). The solid black line shows native disulfide bond and the amino acid residues highlighted in yellow are readily phosphorylated with methods disclosure herein.

FIG. 13 shows amino acid sequence of human amylin peptide (SEQ ID NO: 2).

### DETAILED DESCRIPTION

For the purposes of promoting an understanding of the principles of the present disclosure, references will now be made to the embodiments illustrated in the drawings, and specific language will be used to describe the same. It will nevertheless be understood that no limitation of the scope of this disclosure is thereby intended.

As used herein, the following terms and phrases shall have the meanings set forth below. Unless defined otherwise, all technical and scientific terms used herein have the same meaning as commonly understood to one of ordinary skill in the art.

In the present disclosure the term “about” can allow for a degree of variability in a value or range, for example, within 20%, within 15%, within 10%, within 5%, or within 1% of a stated value or of a stated limit of a range. In the present disclosure the term “substantially” can allow for a degree of variability in a value or range, for example, within 80%, within 85%, within 90%, within 95%, or within 99% of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated references should be considered supplementary to that of this document; for irreconcilable inconsistencies, the usage in this document controls.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “administering” includes all means of introducing the compounds and compositions described herein to the patient, including, but not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles. In some embodiments, administering may also be carried out via an implantable devices.

Solid medicinal forms can comprise inert components and carrier substances, such as calcium carbonate, calcium phosphate, sodium phosphate, lactose, starch, mannitol, alginates, gelatin, guar gum, magnesium stearate, aluminum stearate, methyl cellulose, talc, highly dispersed silicic acids, silicone oil, higher molecular weight fatty acids, (such as stearic acid), gelatin, agar or vegetable or animal fats and oils, or solid high molecular weight polymers (such as polyethylene glycol); preparations which are suitable for oral administration can comprise additional flavorings and/or sweetening agents, if desired.

Liquid medicinal forms can be sterilized and/or, where appropriate, comprise auxiliary substances, such as preservatives, stabilizers, wetting agents, penetrating agents, emulsifiers, spreading agents, solubilizers, salts, sugars or sugar alcohols for regulating the osmotic pressure or for buffering, and/or viscosity regulators. Examples of such additives are tartrate and citrate buffers, ethanol and sequestering agents (such as ethylenediaminetetraacetic acid and its nontoxic salts). High molecular weight polymers, such as liquid polyethylene oxides, microcrystalline celluloses, carboxymethyl celluloses, polyvinylpyrrolidones, dextrans or gelatine, are suitable for regulating the viscosity. Examples of solid carrier substances are starch, lactose, mannitol, methyl cellulose, talc, highly dispersed silicic acids, high molecular weight fatty acids (such as stearic acid), gelatine, agar, calcium phosphate, magnesium stearate, animal and vegetable fats, and solid high molecular weight polymers, such as polyethylene glycol.

Oily suspensions for parenteral or topical applications can be vegetable, synthetic or semisynthetic oils, such as liquid fatty acid esters having in each case from 8 to 22 carbon atoms in the fatty acid chains, for example palmitic acid, lauric acid, tridecanoic acid, margaric acid, stearic acid, arachidic acid, myristic acid, behenic acid, pentadecanoic acid, linoleic acid, elaidic acid, brasidic acid, erucic acid or oleic acid, which are esterified with monohydric to trihydric alcohols having from 1 to 6 carbon atoms, such as methanol, ethanol, propanol, butanol, pentanol or their isomers, glycol or glycerol. Examples of such fatty acid esters are commercially available miglyols, isopropyl myristate, isopropyl palmitate, isopropyl stearate, PEG 6-capric acid, caprylic/capric acid esters of saturated fatty alcohols, polyoxyethylene glycerol trioleates, ethyl oleate, waxy fatty acid esters, such as artificial ducktail gland fat, coconut fatty acid isopropyl ester, oleyl oleate, decyl oleate, ethyl lactate, dibutyl phthalate, diisopropyl adipate, polyol fatty acid esters, inter alia. Silicone oils of differing viscosity, or fatty alcohols, such as isotridecyl alcohol, 2-octyldodecanol, cetylstearyl alcohol or oleyl alcohol, or fatty acids, such as oleic acid, are also suitable. It is furthermore possible to use vegetable oils, such as castor oil, almond oil, olive oil, sesame oil, cotton seed oil, groundnut oil, soybean oil or the like.

Suitable solvents, gelatinizing agents and solubilizers are water or water miscible solvents. Examples of suitable substances are alcohols, such as ethanol or isopropyl alcohol, benzyl alcohol, 2-octyldodecanol, polyethylene glycols, phthalates, adipates, propylene glycol, glycerol, di- or tripropylene glycol, waxes, methyl cellosolve, cellosolve, esters, morpholines, dioxane, dimethyl sulfoxide, dimethylformamide, tetrahydrofuran, cyclohexanone, etc.

Mixtures of gelatinizing agents and film-forming agents are also perfectly possible. In this case, use is made, in particular, of ionic macromolecules such as sodium carboxymethyl cellulose, polyacrylic acid, polymethacrylic acid and their salts, sodium amylopectin semiglycolate, alginic acid or propylene glycol alginate as the sodium salt, gum arabic, xanthan gum, guar gum or carrageenan. The following can be used as additional formulation aids: glycerol, paraffin of differing viscosity, triethanolamine, collagen, allantoin and novantisolic acid. Use of surfactants, emulsifiers or wetting agents, for example of sodium lauryl sulphate, fatty alcohol ether sulphates, di-sodium-N-lauryl-iminodipropionate, polyethoxylated castor oil or sorbitan monooleate, sorbitan monostearate, polysorbates (e.g. Tween), cetyl alcohol, lecithin, glycerol monostearate, polyoxyethylene stearate, alkylphenol polyglycol ethers, cetyltrimethylammonium chloride or mono-/dialkylpolyglycol ether orthophosphoric acid monoethanolamine salts can also be required for the formulation. Stabilizers, such as montmorillonites or colloidal silicic acids, for stabilizing emulsions or preventing the breakdown of active substances such as antioxidants, for example tocopherols or butylhydroxyanisole, or preservatives, such as p-hydroxybenzoic acid esters, can likewise be used for preparing the desired formulations.

Preparations for parenteral administration can be present in separate dose unit forms, such as ampoules or vials. Use is preferably made of solutions of the active compound, preferably aqueous solution and, in particular, isotonic solutions and also suspensions. These injection forms can be made available as ready-to-use preparations or only be prepared directly before use, by mixing the active compound, for example the lyophilizate, where appropriate containing other solid carrier substances, with the desired solvent or suspending agent.

Intranasal preparations can be present as aqueous or oily solutions or as aqueous or oily suspensions. They can also be present as lyophilizates which are prepared before use using the suitable solvent or suspending agent.

Inhalable preparations can present as powders, solutions or suspensions. Preferably, inhalable preparations are in the form of powders, e.g. as a mixture of the active ingredient with a suitable formulation aid such as lactose.

The preparations are produced, aliquoted and sealed under the customary antimicrobial and aseptic conditions.

As indicated above, a compound of the invention may be administered as a combination therapy with further active agents, e.g. therapeutically active compounds useful in the treatment of cancer, for example, prostate cancer, ovarian cancer, lung cancer, or breast cancer. For a combination therapy, the active ingredients may be formulated as compositions containing several active ingredients in a single dose form and/or as kits containing individual active ingredients in separate dose forms. The active ingredients used in combination therapy may be co-administered or administered separately.

It is to be understood that the total daily usage of the compounds and compositions described herein may be decided by the attending physician within the scope of sound medical judgment. The specific therapeutically effective dose level for any particular patient will depend upon a variety of factors, including the disorder being treated and the severity of the disorder; activity of the specific compound employed; the specific composition employed; the age, body weight, general health, gender, and diet of the patient: the time of administration, and rate of excretion of the specific compound employed, the duration of the treatment, the drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg. The dosage may be single or divided, and may be administered according to a wide variety of dosing protocols, including q.d., b.i.d., t.i.d., or even every other day, once a week, once a month, and the like. In each case the therapeutically effective amount described herein corresponds to the instance of administration, or alternatively to the total daily, weekly, or monthly dose.

As used herein, the term “therapeutically effective amount” refers to that amount of active compound or pharmaceutical agent that elicits the biological or medicinal response in a tissue system, animal or human that is being sought by a researcher, veterinarian, medical doctor or other clinicians, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment.

As used herein, the term “therapeutically effective amount” refers to the amount to be administered to a patient, and may be based on body surface area, patient weight, and/or patient condition. In addition, it is appreciated that there is an interrelationship of dosages determined for humans and those dosages determined for animals, including test animals (illustratively based on milligrams per meter squared of body surface) as described by Freireich, E. J., et al., Cancer Chemother. Rep. 1966, 50 (4), 219, the disclosure of which is incorporated herein by reference. Body surface area may be approximately determined from patient height and weight (see, e.g., Scientific Tables, Geigy Pharmaceuticals, Ardley, N.Y., pages 537-538 (1970)). A therapeutically effective amount of the compounds described herein may be defined as any amount useful for effective treatment in clinic. It is appreciated that effective doses may also vary depending on the route of administration, optional excipient usage, and the possibility of co-usage of the compound with other conventional and non-conventional therapeutic treatments.

The term “patient” as used herein includes human beings and non-human animals such as companion animals (dogs, cats and the like) and livestock animals. Livestock animals are animals raised for food production. The patient to be treated is preferably a mammal, in particular a human being.

In some embodiments, the method disclosed herein may find applications in other biological active peptides, such as calcitonin gene peptide superfamily, insulin and insulin-related peptide hormone superfamily. Calcitonin gene peptide superfamily includes human calcitonin, amylin, calcitonin gene related peptide (CGRP), and adrenomedullin.

Parts of this disclosure has been published at Renawala et al., Biophysical Journal, 2020, Vol. 120 (1), pp. 86-100, entitled “Fibrillation of Human Calcitonin and Its Analogs: Effects of Phosphorylation and Disulfide Reduction”, the contents of which are incorporated herein by reference in its entirety.

In some illustrative embodiments, this disclosure relates to a modified peptide, or a pharmaceutically acceptable salt thereof, wherein said modified peptide has no or substantially reduced fibrillation whereby said modified peptide produces a stable aqueous solution, due to said modification.

In some illustrative embodiments, this disclosure relates to a modified peptide, or a pharmaceutically acceptable salt thereof, wherein said modified peptide has no or substantially reduced fibrillation whereby said modified peptide produces a stable aqueous solution, due to said modification, wherein said modification is phosphorylation of one or more side chains of amino acid residues of said peptide.

In some illustrative embodiments, this disclosure relates to a modified peptide, or a pharmaceutically acceptable salt thereof, wherein said modified peptide has no or substantially reduced fibrillation whereby said modified peptide produces a stable aqueous solution, due to said modification, wherein said modified peptide comprises the amino acid sequence of SEQ ID NO: 1 (human calcitonin peptide), or a substantial fragment thereof, modified such that said modified peptide has no or substantially reduced fibrillation whereby said modified peptide produces a stable aqueous solution.

In some illustrative embodiments, this disclosure relates to a modified peptide, or a pharmaceutically acceptable salt thereof, wherein said modified peptide has no or substantially reduced fibrillation whereby said modified peptide produces a stable aqueous solution, due to said modification, wherein said modified peptide comprises the amino acid sequence of SEQ ID NO: 2 (human amylin peptide), or a substantial fragment thereof, modified such that said modified peptide has no or substantially reduced fibrillation whereby said modified peptide produces a stable aqueous solution.

In some illustrative embodiments, this disclosure relates to a modified peptide, or a pharmaceutically acceptable salt thereof, wherein said modified peptide has no or substantially reduced fibrillation whereby said modified peptide produces a stable aqueous solution, due to said modification, wherein said modification of SEQ ID NO: 1 is carried out on the side chain of Ser5, Thr6, Thr11, Tyr12, Thr13, His20, Thr21, Thr25, or a combination thereof.

In some illustrative embodiments, this disclosure relates to a modified peptide, or a pharmaceutically acceptable salt thereof, wherein said modified peptide has no or substantially reduced fibrillation whereby said modified peptide produces a stable aqueous solution, due to said modification, wherein said modification of SEQ ID NO: 2 is carried out on the side chain of Thr4, Thr6, Thr9, His18, Ser19, Ser20, Ser28, Ser29, Thr30, Ser34, Thr36, Tyr37, or a combination thereof.

In some other illustrative embodiments, this disclosure relates to a pharmaceutical composition comprising:
a. a phosphorylated peptide or a pharmaceutically acceptable salt thereof, wherein said modified peptide has no or substantially reduced fibrillation whereby said phosphorylated peptide produces a stable aqueous solution, due to said phosphorylation; and
b. one or more pharmaceutically acceptable diluents, excipients or carriers.

In some other illustrative embodiments, this disclosure relates to a pharmaceutical composition as disclosed herein, wherein said modified peptide comprises the amino acid sequence of SEQ ID NO: 1 (human calcitonin), or a substantial fragment thereof, modified such that said modified peptide has no or substantially reduced fibrillation whereby said modified peptide produces a stable aqueous solution.

In some other illustrative embodiments, this disclosure relates to a pharmaceutical composition as disclosed herein, wherein said modified peptide comprises the amino acid sequence of SEQ ID NO: 2 (human amylin peptide), or a substantial fragment thereof, modified such that said modified peptide has no or substantially reduced fibrillation whereby said modified peptide produces a stable aqueous solution.

In some other illustrative embodiments, this disclosure relates to a pharmaceutical composition as disclosed herein, wherein said modification of SEQ ID NO: 1 is carried out on the side chain of Ser5, Thr6, Thr11, Tyr12, Thr13, His20, Thr21, Thr25, or a combination thereof.

In some other illustrative embodiments, this disclosure relates to a pharmaceutical composition as disclosed herein, wherein said modification of SEQ ID NO: 2 is carried out on the side chain of Thr4, Thr6, Thr9, His18, Ser19, Ser20, Ser28, Ser29, Thr30, Ser34, Thr36, Tyr37, or a combination thereof.

Yet in some other illustrative embodiments, this disclosure relates to a method for treating a patient of osteoporosis comprising the step of administrating a therapeutically effective amount of a pharmaceutical composition as disclosed herein.

Yet in some other illustrative embodiments, this disclosure relates to a method for treating a patient suffering from pain comprising the step of administrating a therapeutically effective amount of a pharmaceutical composition as disclosed herein.

Yet in some other illustrative embodiments, this disclosure relates to a method for treating Paget's disease comprising the step of administrating a therapeutically effective amount of a pharmaceutical composition as disclosed herein.

Yet in some other illustrative embodiments, this disclosure relates to a method for treating a patient of hypercalcemia comprising the step of administrating a therapeutically effective amount of a pharmaceutical composition as disclosed herein.

Yet in some other illustrative embodiments, this disclosure relates to a method for promoting integration of bone and a metal implant post-surgery comprising the step of administrating a therapeutically effective amount of a pharmaceutical composition as disclosed herein.

Materials and Methods

Research grade human calcitonin and phosphorylated human calcitonin derivatives were purchased from ABclonal Technology (Woburn, Mass.). Deuterium oxide (D₂O, 99.9%) was purchased from Cambridge Isotope Laboratories (Andover, Mass.). Thioflavin T was procured from Abcam (Cambridge, Mass.) and Tris 2-carboxyethyl phosphine (TCEP) hydrochloride was purchased from Sigma-Aldrich (St. Louis, Mo.). 96-well non-binding, black wall, clear bottom microtiter plates (#655906) for fibrillation kinetics studies were purchased from Greiner Bio-One (Frickenhausen, Germany). The microtiter plates were covered with MicroAmp Optical Adhesive Film (ThermoFisher Scientific, Waltham, Mass.). Poroszyme Immobilized Pepsin Bulk Media was purchased from Applied Biosystems (Foster City, Calif.) and packed into a high-performance liquid chromatography column (50×2.1 mm, Grace Davison Discovery Sciences, Deerfield, Ill.). Mass spectrometry grade water, acetonitrile (ACN) and formic acid (FA) were from ThermoFisher Scientific (Waltham, Mass.). All other chemicals used were at least reagent grade and used as received.

Sample Preparation by Dialysis and Lyophilization

The peptides, human calcitonin (hCT) and phosphorylated human calcitonin derivatives (phCT) had trifluoroacetate salt impurities from the manufacturing process, which were removed by dialysis. The peptides were dissolved in deionized (di) water at 2 mg/mL concentration and dialyzed using 2 kDa MWCO Slide-A-Lyzer Dialysis Cassettes (ThermoFisher Scientific), with di-water as the dialysis medium for a duration of 6 h under refrigerated conditions, with a change in media every 2 h. After dialysis, the peptide solution was filtered through a 0.1 m PVDF filter and transferred to lyophilization vials (size 2R, DWK Life Sciences, Millville, N.J.). The peptide solution was freeze-dried using a pilot scale LyoStar 3 lyophilizer (SP Scientific, Warminster, Pa.) to obtain dried and purified peptide powder, which was stored at −80° C. until use. The lyophilization process was initiated by precooling the shelf to 5° C. followed by freezing at −40° C. for 2 h, primary drying at −35° C. for 24 h under vacuum (70 mTorr), and secondary drying at 25° C. for 6 h under vacuum (70 mTorr).

Reduction of Disulfide Bond

Reduction of the intramolecular 1-7 Cys-Cys disulfide bond in human calcitonin (hCT) was carried out by using Tris 2-carboxyethyl phosphine (TCEP) hydrochloride as reducing agent. hCT was dissolved in di-water at 2 mg/mL followed by addition of 100 mM TCEP stock solution to give a final TCEP concentration of 5 mM. The solution was allowed to stand for 15 min to complete the reduction reaction, which was confirmed by the absence of native hCT peak using mass spectrometry. The reduced hCT solution was then dialyzed to remove dissolved salt impurities and TCEP. The solution was then lyophilized to obtain dried and purified reduced human calcitonin (rhCT) as described above.

Thioflavin-T Fluorescence Measurement

The fibrillation kinetics of hCT, rhCT and phCT was monitored by measuring the increase in fluorescence intensity upon thioflavin T (ThioT) binding to peptide fibrils. The ThioT stock solution (2.5 mM) was prepared in di-water and diluted 10-fold with buffer to give a working solution (250 μM). The peptides were weighed and dissolved in cold 20 mM sodium phosphate buffer pH 7.4, to a concentration of 100 μM (˜0.34 mg/mL). The peptide solution was quickly transferred to a 96-well microtiter plate and placed in a Synergy Neo2 Multi-Detection microplate reader (BioTek Instruments, Winooski, Vt.). 5 μL of ThioT working solution (250 μM) was added to 245 μL of peptide solution in each well to give a final ThioT concentration of 5 μM per well. At each time point, six replicate measurements were performed for each sample. Fibrillation was carried out for 12 or 24 h at 25° C. with continuous shaking at 731 cpm. The fluorescence intensity of ThioT was bottom-read at an interval of 10 or 15 min, at excitation and emission wavelengths of 440 nm and 482 nm, respectively.

The ThioT fluorescence data were fitted to the Boltzmann sigmoidal function as given below, using GraphPad Prism version 7.03 (GraphPad Software, La Jolla, Calif.).

$$Y = {A}_{2} + \frac{{A}_{1} - {A}_{2}}{1 + {e}^{( x - {x}_{o} ) / d {x}^{′}}} \qquad ( 1 )$$

where A₁ is the minimum signal, A₂ is the maximum signal, xₒ is the time at which the change in signal is 50% (t₅₀) and dx′ is the slope of growth phase.¹ The lag time was determined either by sigmoidal fit to the fibrillation kinetics curve or using onset point determination. In sigmoidal fits, the lag time was determined as xₒ−2 dx′ using parameters obtained from nonlinear fit of the data to Eq. 1. For data that did not fit satisfactorily to the Boltzmann sigmoidal function, the lag time was determined from the intersection of tangents to the lag phase and growth phase curves, which was considered to be the onset point of ThioT fluorescence signal.² The lag times determined from the sigmoidal curve fitting and the onset point determination method were within 3% of one another. In cases where sigmoid curve fitting could not be achieved, the t₅₀ was estimated from the raw data as the time when the signal intensity reached half of the observed maximum value.

Turbidity Measurement

Fibrillation kinetics of hCT, rhCT and phCT was followed by measuring the increase in solution turbidity due to formation of soluble oligomeric species and insoluble fibrils. The peptides were weighed and dissolved in cold 20 mM sodium phosphate buffer pH 7.4, to a concentration of 100 μM (˜0.34 mg/mL). 250 μL of peptide solution was quickly transferred to each well of a 96-well microtiter plate and placed in the Synergy Neo2 Multi-Detection microplate reader (BioTek Instruments). At each time point, six replicate measurements were performed for each sample. Fibrillation was carried out for 12 or 24 h at 25° C. with continuous shaking at 731 cpm. Solution turbidity was measured by UV absorbance at 340 nm at 10 or 15-min read intervals. The turbidity data was fitted to the Boltzmann sigmoidal function (Eq. 1) and analyzed as described above.

CD Spectroscopy

Changes in the secondary structure of hCT and phCT were monitored by CD spectroscopy using a Jasco J-815 spectrometer (JASCO Analytical Instruments, Easton, Md.). Solution samples were prepared by dissolving lyophilized peptides in cold 20 mM sodium phosphate buffer pH 7.4, at a concentration of 100 μM (˜0.34 mg/mL) and allowed to fibrillate in 96-well microtiter plates placed inside a BioTek Synergy Neo2 plate reader as described above. At predetermined time intervals, samples were withdrawn for CD analysis. CD spectra were recorded in a quartz cuvette with 1 mm pathlength, scanned from 190 nm to 260 nm in the far UV range, at a scan speed of 50 nm/min at 20° C. Each spectrum was reported as an average of three scans.

Amide HDX-MS

HDX-MS for Monomeric hCT—As a control, the rate and extent of deuterium uptake were measured for hCT under conditions when fibrillation did not occur. A 100 μM (˜0.34 mg/mL) hCT solution was freshly prepared by dissolving lyophilized hCT in 20 mM sodium phosphate buffer pH 7.4 at room temperature. Deuteration was initiated by 10-fold dilution of the hCT solution with D₂O (99.9%), resulting in a final concentration of 90% D₂O (v/v) and 10 μM hCT. The deuterated peptide solution was kept undisturbed at room temperature for the duration of the study. At various times after the initiation of deuteration (0.5, 1.5, 3, 5, 7.5, 10, 20, 30, 60, and 120 min), 45 μL of the sample was withdrawn and the exchange reaction quenched by addition of 45 μL of ice-cold quench buffer containing 5 M urea, 5% v/v methanol and 0.2% v/v formic acid in MS grade water (pH 2.5). The quenched samples were flash frozen by immersion in liquid nitrogen and stored at −80° C. until analysis.

Pulsed HDX-MS for hCT and phCT During Fibrillation—Decreases in deuterium incorporation in pulsed hydrogen deuterium exchange studies (HDX-MS) have been used to measure fibrillation kinetics.¹ To monitor the fibrillation of hCT and phCT using HDX-MS, peptide solutions were allowed to fibrillate in 96-well microtiter plates placed inside a Synergy Neo2 Multi-Detection microplate reader (BioTek Instruments) under the same experimental conditions used for the ThioT and turbidity studies described above. The sample solutions were prepared by dissolving lyophilized peptide powder in cold 20 mM sodium phosphate buffer pH 7.4, to a concentration of 100 μM (˜0.34 mg/mL). 250 μL of peptide solution was quickly transferred to each well of a microtiter plate. At predetermined time intervals, samples were withdrawn from the microplate reader. The time needed for sampling was negligible (˜1 min) compared to overall duration of fibrillation. For hCT, fibrillation in individual wells of the microtiter plate was monitored by sampling every time point (0-1440 min) from same wells. Three replicate samples were withdrawn at each time point for intact and fragment level analysis. A 2 min pulse deuterium labelling period was initiated by diluting a 5 μL sample of fibrillating peptide in 45 μL of D₂O (99.9%), resulting in a final concentration of 90% D₂O (v/v) and 10 μM peptide. The pulse-labeled samples were quenched and stored at −80° C. until analysis.

HDX-MS Instrumentation—For both monomeric and pulsed HDX-MS studies, deuterium uptake was measured using a high-performance liquid chromatography mass spectrometry (LC-MS) system (Agilent 6520 QTOF, Agilent Technologies, Santa Clara, Calif.). The system was equipped with a custom-built refrigeration unit housing the LC system capable of maintaining the low temperatures (˜2° C.) required to minimize back exchange of deuterated samples. The deuterated samples were quickly thawed and ˜250 pmole of calcitonin was injected into the LC-MS system. The samples were retained on a peptide microtrap (Michrom Bioresources, Auburn, Calif.) and desalted with 0.1% FA in water for 1.7 min at a flow rate of 0.2 m/min. The peptide was then eluted in 4.5 min on an analytical column (Zorbax 300SB-C18; Agilent Technologies, Santa Clara, Calif.) using a gradient of ACN (25-59%), water and 0.1% FA at a flow rate of 50 μL/min. Mass spectra were collected over the m/z range of 100-1700. To measure the deuterium uptake at the fragment level, proteolytic digestion was carried out by injecting ˜250 pmole of calcitonin into an in-line immobilized pepsin column, maintained at 25° C. by housing it in a heating oven placed inside the cold box. Online digestion was carried out for 1.7 min in water containing 0.1% FA, at a flow rate of 0.2 mg/mL. The calcitonin fragments were desalted and eluted on a C18 analytical column as described above. The pepsin column, peptide microtrap and analytical column were located within the refrigeration unit.

HDX-MS Data Analysis—For HDX-MS analysis at fragment level, a list of peptide fragments was generated from each undeuterated control using MassHunter Workstation software equipped with the Bioconfirm software package (B.03.01, Agilent Technologies). Peptides identified using undeuterated controls were mapped onto subsequent deuteration experiments using HDExaminer software (Version 2.0, Sierra Analytics, Modesto, Calif.). For fibrillating peptides showing a bimodal distribution of deuterium uptake, the percent deuteration was calculated by the software as a weighted average of the deuterium uptake in the protected and more accessible populations. The deuterium uptake values were not subjected to back exchange correction. All values are reported as the mean of three independent HDX experiments.

For freshly prepared hCT solutions, the extent of deuteration as a function of time was fitted to a mono-exponential association model (Eq. 2) for deuterium uptake.

D=Dₘₐₓ(1−e^{-kt}) (2)

where Dmax is the maximum deuteration, k is the exchange rate constant and t is deuteration time (min).

The deuterium uptake data for fibrillating hCT were fitted to the Boltzmann sigmoidal function (Eq. 1). Data analysis was performed using GraphPad Prism version 7.03.

Fibrillation Kinetics using Fluorescence and Turbidity Measurements

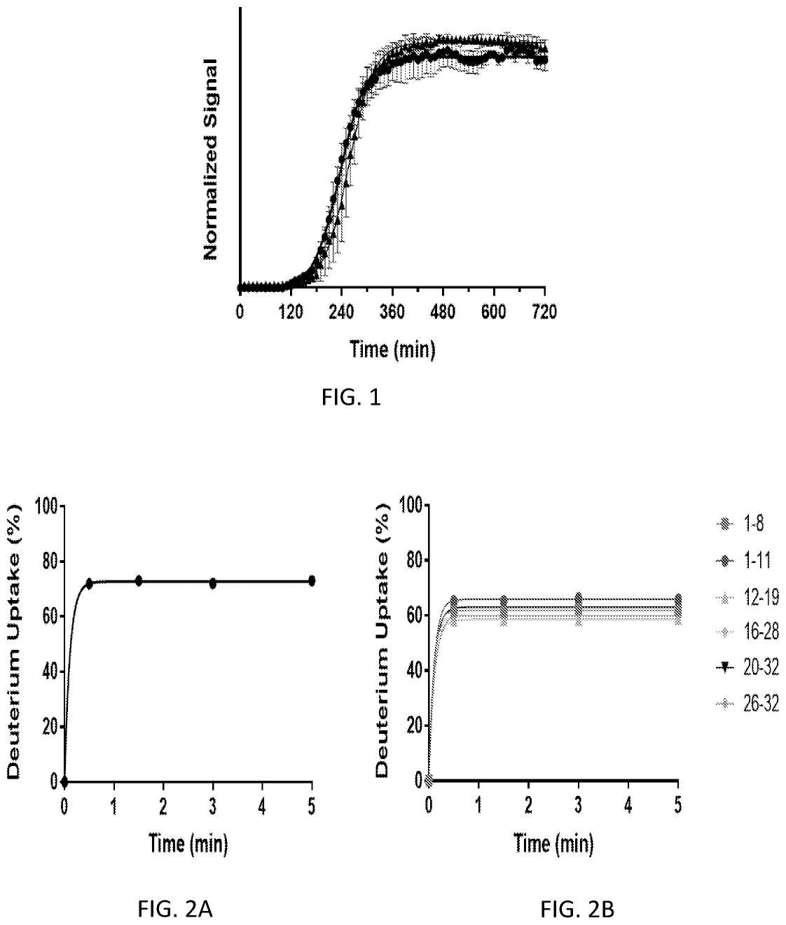

The fibrillation kinetics of hCT were monitored by recording the fluorescence emission of ThioT dye and changes in solution turbidity. ThioT is a fluorescent dye frequently used to detect fibril formation, giving an increased fluorescent signal on binding to the hydrophobic pockets of 3-sheet rich fibrils.³ During fibrillation, an increase in ThioT fluorescence was observed consistent with the formation of 3-sheet rich fibrils (FIG. 1). An increase in solution turbidity was also observed, consistent with an increase in the number and/or size of particles (FIG. 1). For both ThioT and turbidity measurements, fibrillation kinetics showed sigmoidal behavior, with lag, growth and equilibrium phases characteristic of a nucleation-dependent aggregation process.² For hCT, the fibrillation lag times for ThioT and turbidity measurements were 172±15 min and 198±20 min, respectively and the t₅₀ values were 236±8 min and 257±15 min, respectively (Table 1). There was no significant difference between values measured by ThioT fluorescence and those measured by turbidity.

Amide HDX-MS

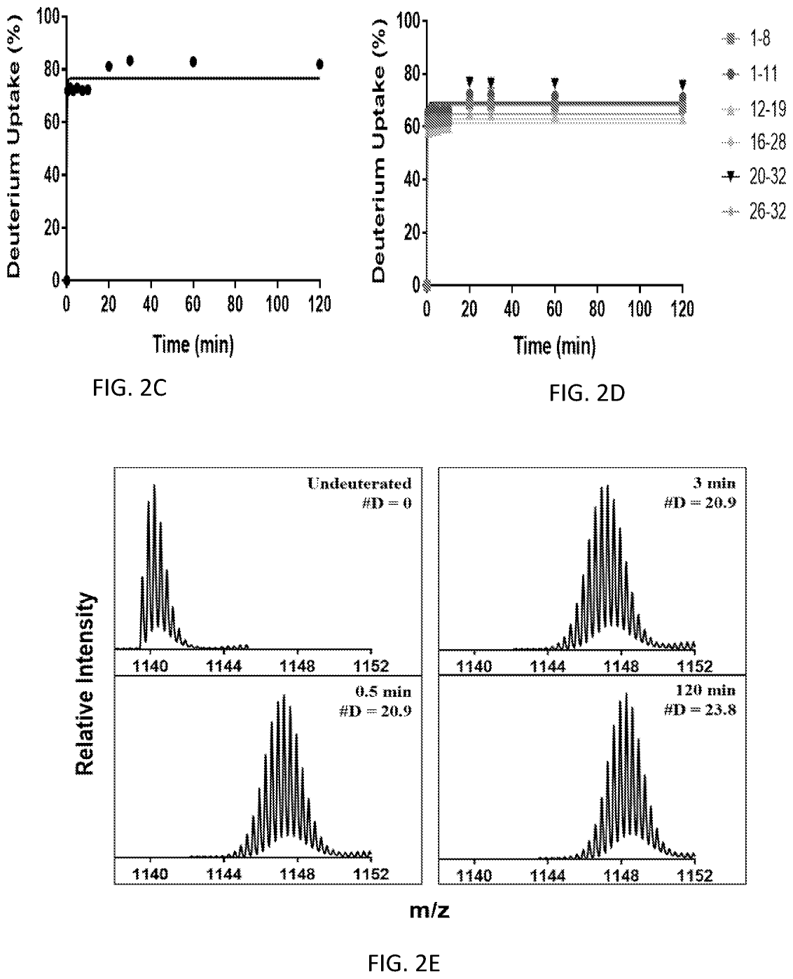

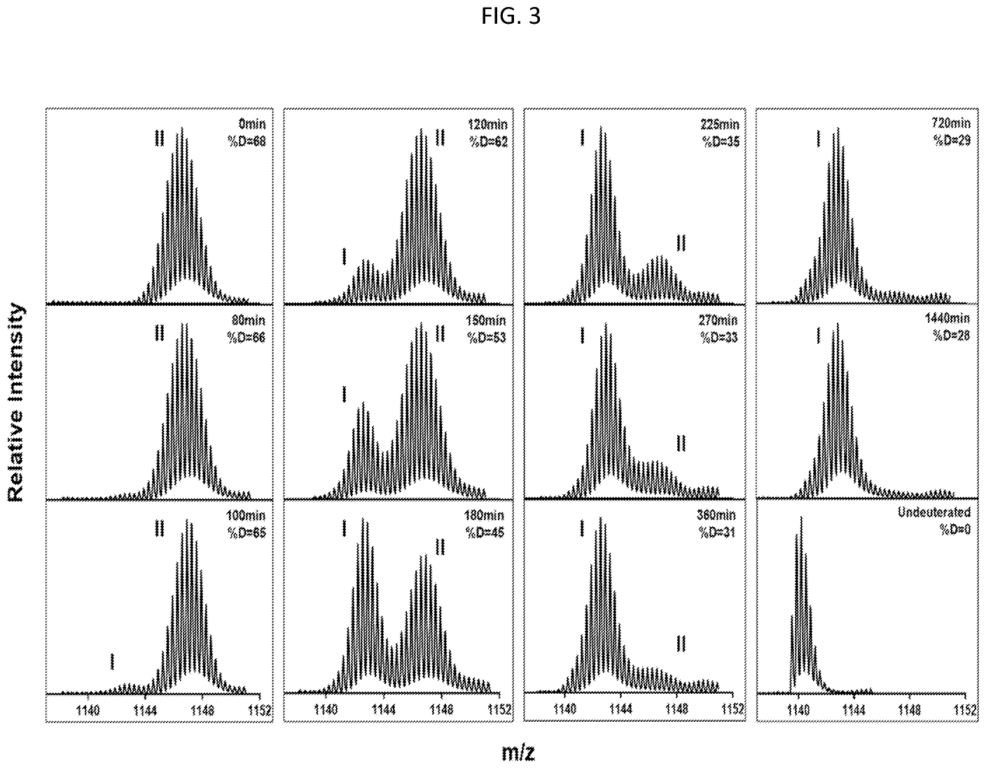

Amide HDX-MS for Monomeric hCT—HDX-MS was performed at both intact and fragment level to determine the extent of deuterium uptake in monomeric hCT, serving as a control for pulsed deuteration studies of fibrillating hCT. An increase in deuterium uptake with time was observed for hCT at the intact level (FIG. 2A, FIG. 2C), as expected. Percent deuterium uptake reached 73% of the theoretical maximum in less than 2 min (FIG. 2A) and after 20 min of D₂O exposure, the deuterium uptake increased to 82% of the theoretical maximum (FIG. 2C). The latter value suggests an upper limit for back exchange in this system of ˜18%, in agreement with previous HDX studies of unstructured peptides in our lab.⁴ The mass spectrum showed a single peak for deuterated hCT monomer, with no new peak or significant peak broadening at any time point (FIG. 2E). The percent deuterium uptake data for monomeric hCT were fitted to a mono-exponential association model (Eq. 2) and showed a half-life (t₅₀) of ˜5 sec, which is in agreement with the observed near-maximum deuterium uptake for monomeric hCT at the first measured time point of 30 sec (FIG. 2A) and indicates that a 2-min pulse labeling in fibrillation studies is sufficient to deuterate native hCT.

Deuterium uptake of monomeric hCT was also monitored at the fragment level using pepsin digestion followed by LC/MS analysis. A total of six fragments (1-8, 1-11, 12-19, 16-28, 20-32, and 26-32) were selected which showed strong and reproducible MS signals (FIG. 2B), providing 100% sequence coverage with some overlap. The six fragments showed a maximum percent deuterium uptake of 62% (1-8), 66% (1-11), 59% (12-19), 60% (16-28), 63% (20-32), and 60% (26-32), in less than 2 min of D₂O exposure. After 20 min, the percent deuterium levels increased slightly in all six fragments (FIG. 2D).

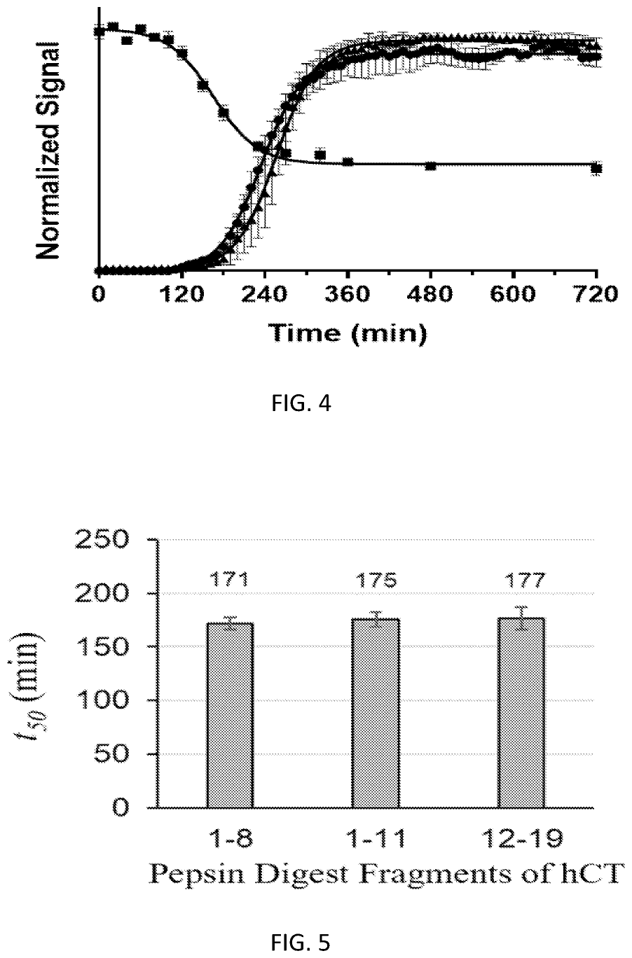

Amide HDX-MS for Fibrillating hCT at Intact Level—At the initiation of the fibrillation experiment (t=0 min), hCT showed a single mass envelope with little peak broadening relative to the undeuterated control (FIG. 3). This peak (Peak II) corresponds to hCT that is highly solvent exposed and deuterated at the level measured under monomeric conditions (% D=68). Similar envelopes were observed until 100 min of fibrillation (FIG. 3), corresponding with the lag phase measured in ThioT and turbidity studies (FIG. 4). After 100 min of fibrillation, a new peak (Peak I) was observed with lower m/z indicating greater protection from exchange (FIG. 3). In HDX-MS, greater protection from exchange is typically due to greater conformational stability and/or intermolecular association.⁵ Here, we infer that Peak I corresponds to hCT oligomers or proto fibrils. As fibrillation proceeds, the intensity of Peak I increases and the intensity of Peak II decreases, with an overall reduction in deuteration levels (FIG. 3, FIG. 4). At 360 min, Peak II appeared as a shoulder, which suggests a near complete shift in population from monomeric to oligomeric hCT. After 1440 min of fibrillation, the percent deuterium uptake was 28%, suggesting that some solvent accessible regions remain in these more mature hCT fibrils (FIG. 3).

The deuterium uptake at any time point is the weighted average of the number of deuterons in both the protected and solvent-accessible populations. The amount of deuterium uptake was plotted as a function of fibrillation time and fitted to a Boltzmann sigmoid function (Eq. 1) (FIG. 4). The lag time of fibrillation by HDX-MS (lag time=104±7 min) was significantly shorter than for ThioT (lag time=172±15 min) and turbidity (lag time=198±20 min) measurements, in agreement with the detection of a more protected population (peak I) at 100 min by HDX-MS (FIG. 3, Table 1). With regards to the half-life of deuterium uptake, a significantly shorter t₅₀ value (t₅₀=165±7 min) was observed for HDX-MS than for ThioT (t₅₀=236±8 min) or turbidity (t₅₀=257±15 min) measurements, suggesting that intermolecular interactions and structural changes detected by HDX precede the changes detected by other methods (Table 1).

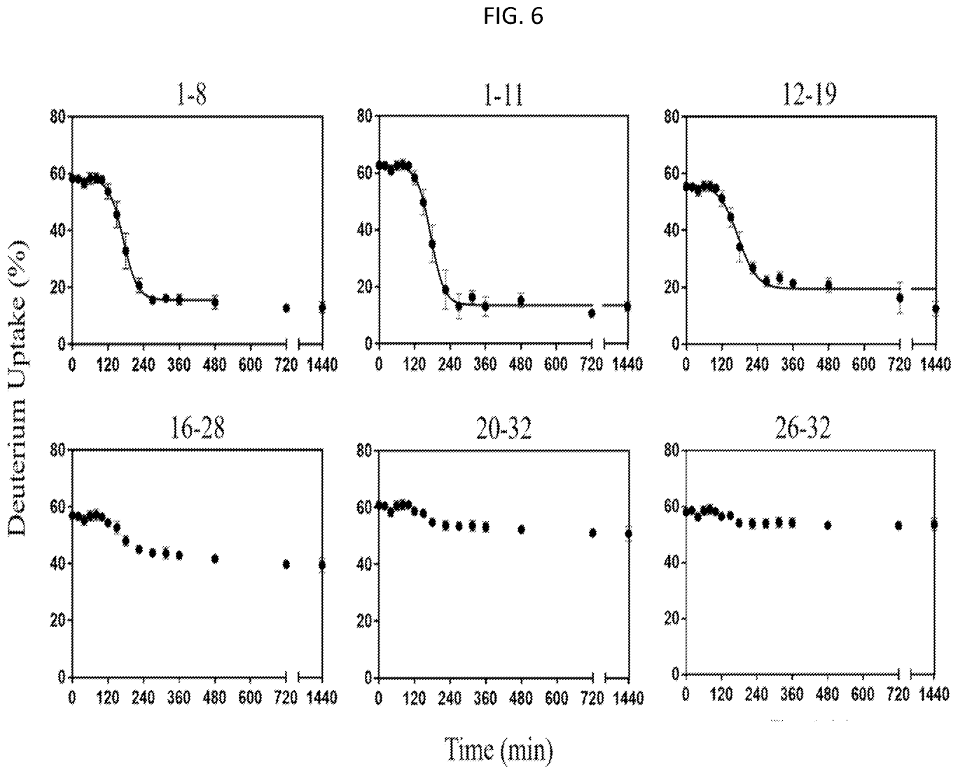

Amide HDX-MS for Fibrillating hCT at Fragment Level—Fibrillating hCT was subjected to HDX-MS with pepsin digestion to identify the regions that show protection from deuteration during the early stages of fibril formation. Deuterium uptake of six hCT fragments was monitored, providing 100% sequence coverage with some overlap (FIG. 6). The average mass of native hCT (3418.1004 g/mol), as measured by mass spectrometry, indicated that the 1-7 Cys-Cys disulfide bond at the N-terminus remained intact during fibrillation and MS analysis. During the initial 100 min of fibrillation, no significant change was observed in the deuteration levels of the six hCT fragments (FIG. 6), consistent with HDX results at the intact level (FIG. 4). After 100 min, the rate and extent of deuterium uptake varied among fragments from different regions of the hCT sequence. Overlapping fragments 1-8 and 1-11, from the N-terminus, and fragment 12-19 all showed a decrease in deuterium uptake with fibrillation time. This decrease was significantly greater than in fragments 20-32 and 26-32 from the C-terminus (FIG. 6). After 1440 min, C-terminal fragments 20-32 and 26-32 showed 51% and 54% deuterium uptake, respectively, decreasing from an initial value of ˜60% (FIG. 6). Deuterium incorporation in the N-terminal fragments 1-8 and 1-11, and in the central fragment 12-19, showed greater decreases to 16%, 13%, and 22% deuterium incorporation, respectively, after 360 min of fibrillation time (FIG. 6). Fragment 16-28, which contains overlapping residues from fragments 12-19 and 20-32, showed intermediate behavior (FIG. 6).

The deuteration kinetics for fragments 1-8, 1-11, and 12-19 were fitted to a Boltzmann sigmoid function (Eq. 1). This analysis was not applied to fragments 16-28, 20-32, and 26-32, which showed a small overall change in deuterium uptake (FIG. 6). The half-lives of deuterium uptake for fragments 1-8, 1-11, and 12-19 did not show significant differences (FIG. 5). For the N-terminal (1-11) and central (12-19) regions, both the relatively rapid decrease in deuterium incorporation and significant decrease in deuteration after 360 min suggest that this region is involved in early stages of hCT fibrillation (FIG. 6). In contrast, for the C-terminal region (20-32 and 26-32), the relatively slow and limited decrease in deuterium incorporation, even after 1440 min, suggest that this region does not participate directly in fibril growth and maturation under these conditions (FIG. 6).

Fibrillation Kinetics of Phosphorylated hCT Derivatives (phCT)

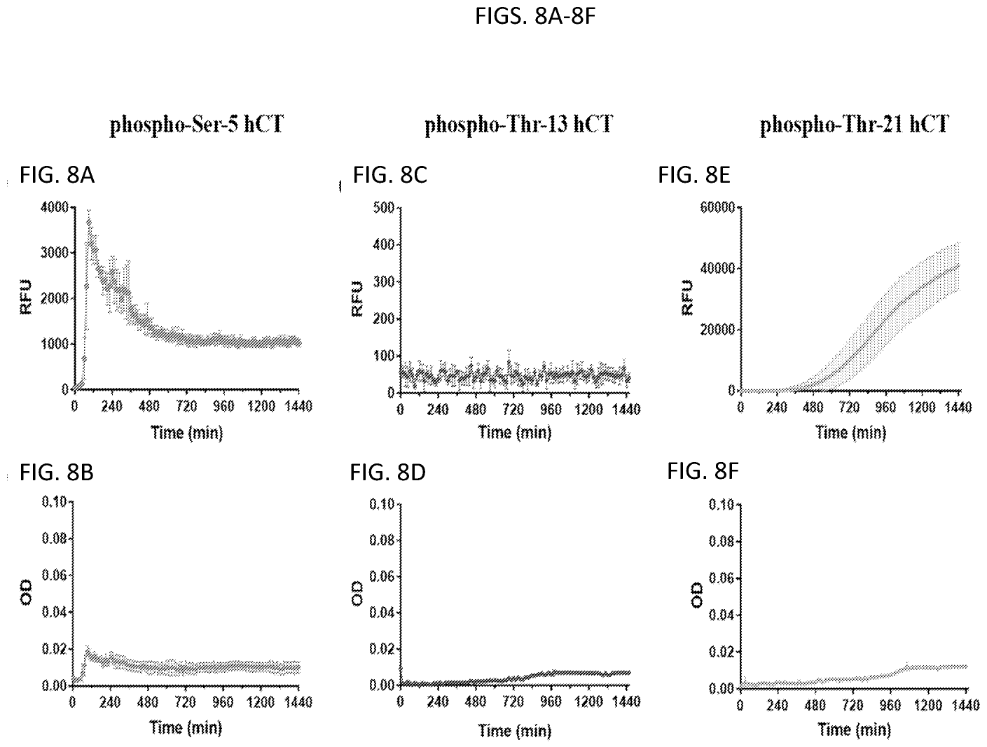

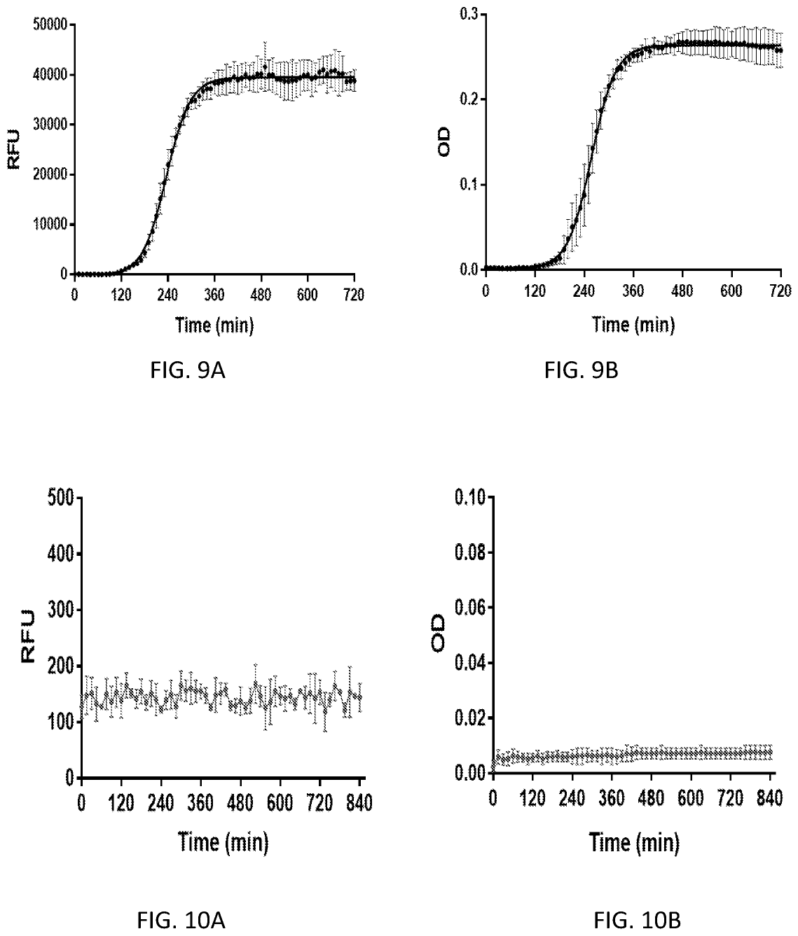

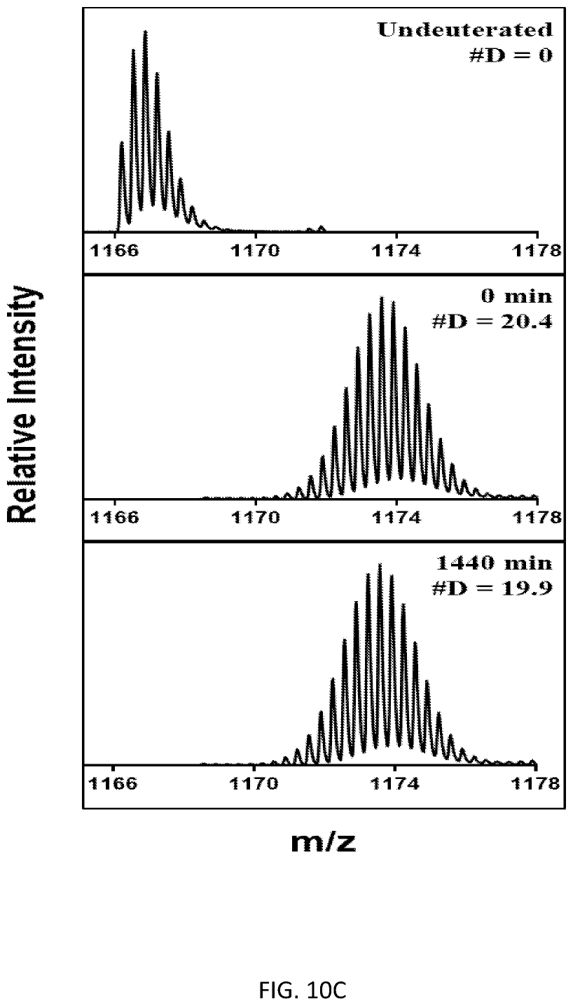

Three phosphorylated human calcitonin (phCT) derivatives were monitored for fibrillation under stressed conditions by ThioT fluorescence and turbidity measurements (FIG. 8). The derivatives showed differences in fibrillation behavior that depended on the site of phosphorylation. An increase in ThioT fluorescence was observed with time for both phospho-Ser-5 hCT and phospho-Thr-21 hCT, consistent with the formation of fibrils (FIG. 8). Phospho-Thr-13 hCT did not show a change in ThioT fluorescence under these conditions, suggesting that fibrillation has been inhibited (FIG. 8). Although the lag time of fibrillation by ThioT was shorter for phospho-Ser-5 hCT (lag time=58±8 min) than for native hCT (lag time=172±15 min) (Table 2), the ThioT signal intensity was ˜10-fold weaker for phospho-Ser-5 hCT than for native hCT (FIG. 8A, FIG. 9A). The lower ThioT signal intensity for phospho-Ser-5 hCT suggests the formation of oligomeric intermediates and/or protofibrils which are known to interact more weakly with ThioT than mature fibrils.⁶ Phospho-Thr-21 hCT showed a much greater fibrillation lag time (lag time=568±95 min) but with ThioT signal intensity similar to native hCT (FIG. 8E, FIG. 9A). The growth of fibrils in phospho-Thr-21 hCT did not plateau in 24 h, as observed by ThioT fluorescence (FIG. 8E). Turbidity measurements for phospho-Ser-5 hCT showed a significantly lower signal intensity compared to native hCT (FIG. 8B, FIG. 9B); on visual inspection a thin translucent film was observed at the bottom of the 96-well plates after 24 h. In contrast, solid dense deposits were observed for hCT fibrillation. There were no significant changes in the turbidity signals for phospho-Thr-13 or phospho-Thr-21 hCT samples (FIGS. 8D, F) and both solutions remained clear on visual inspection. For phospho-Thr13 hCT, fibrillation studies carried out at greater shaking speed of 1096 cpm for 14 h did not show any change in both ThioT and turbidity signals (FIGS. 10A-10B). The weaker ThioT and turbidity intensities along with the nature of oligomeric deposits, suggest that the phosphorylation of Ser5 interferes with fibrillation but does not prevent it. For phospho-Thr-21 hCT, the greater lag time and incomplete growth phase suggest that phosphorylation of Thr21 delays fibril formation and growth, but again does not prevent it entirely. Phosphorylation at Thr13 results in complete inhibition of fibrillation as observed by ThioT and turbidity measurements.

Stability of Phospho-Thr-13 hCT by Amide HDX-MS

HDX-MS at intact level was performed for phospho-Thr-13 hCT samples stressed under fibrillating conditions (25° C., continuous shaking at 731 cpm) for 1440 min. At the start of fibrillation studies (t=0 min), a single peak was observed consistent with monomeric phospho-Thr-13 hCT (FIG. 10C). There was no peak broadening or change in peak position observed after 1440 min (FIG. 10C). The deuterium uptake of phospho-Thr-13 hCT did not change significantly between 0 min and 1440 min and the mass spectrum at 1440 min was consistent with monomeric phospho-Thr-13 hCT (FIG. 10C), indicating the absence of oligomers or fibrillar intermediates.

Secondary Structure Change During Fibrillation by CD Spectroscopy

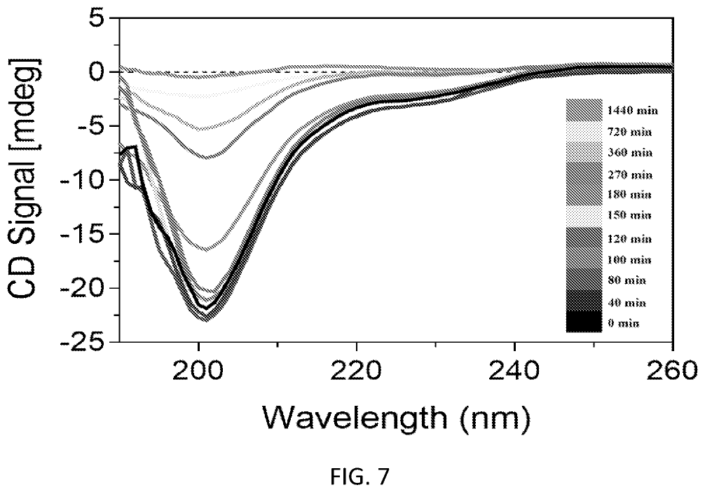

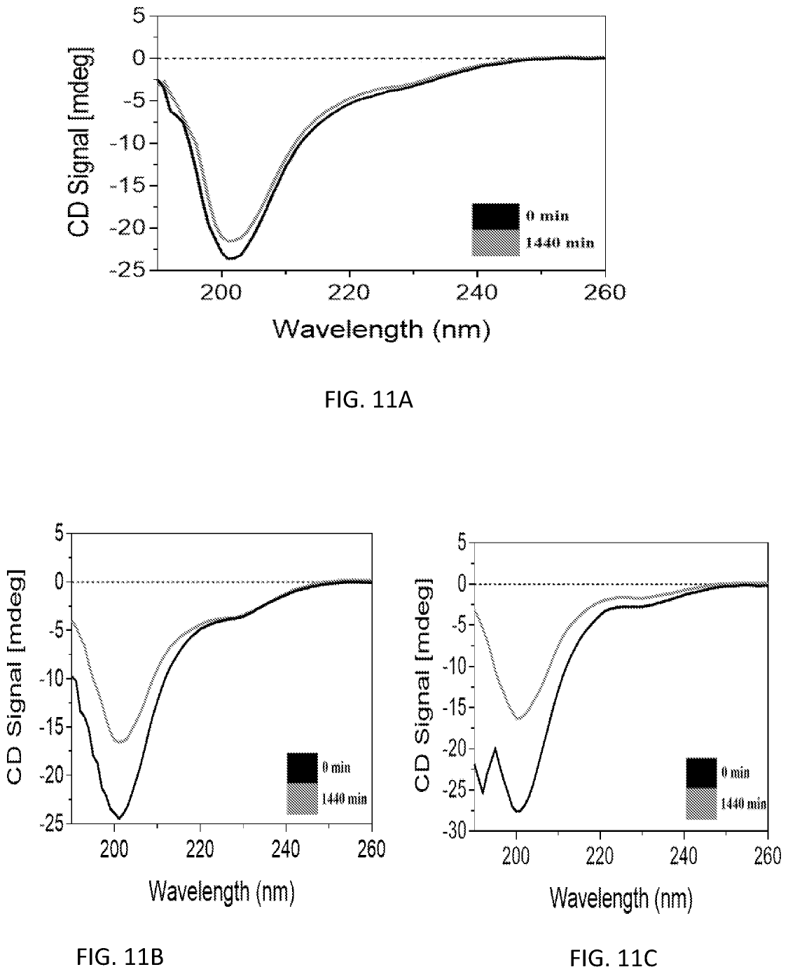

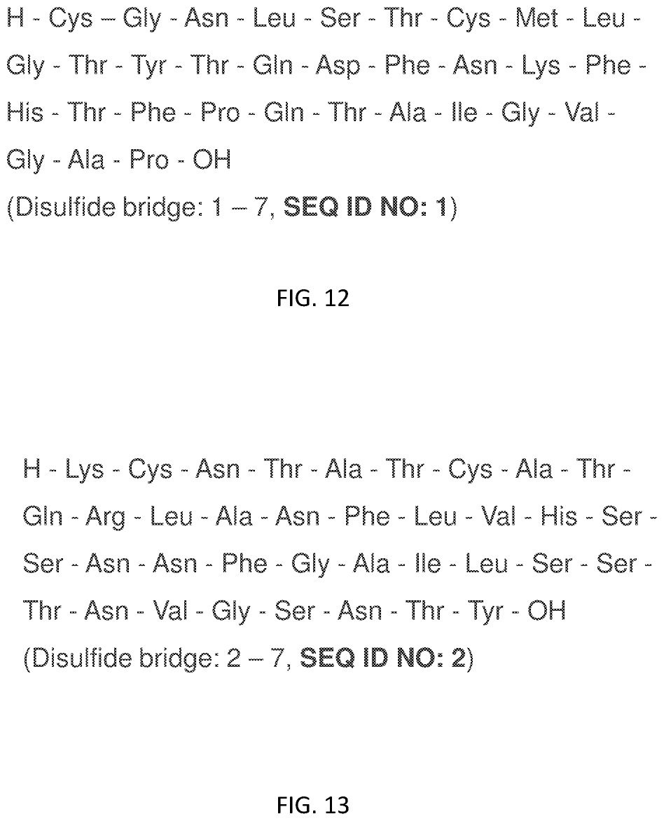

All the hCT analogs showed CD spectra consistent with random coil structures at time zero, as expected (FIG. 7 and FIG. 11).⁷ hCT showed similar random coiled spectra until 150 min of fibrillation time (FIG. 7). After 150 min, CD spectra for hCT began to show reduction of negative intensity followed by a complete loss in signal at around 1440 min (FIG. 7), consistent with the loss in monomer concentration and formation of insoluble fibrils. The three phosphorylated human calcitonin (phospho-hCT) derivatives also showed CD spectra consistent with a randomly coil (FIG. 11). There was no loss of CD signal during the course of fibrillation for phospho-Thr-13, which showed nearly identical spectra at 0 and 1440 min (FIG. 11A). Both phospho-Ser-5 hCT and phospho-Thr-21 hCT showed some reduction in the random coil signal by 1440 min (FIGS. 11B-11C).

TABLE 1
Fibrillation kinetic parameters (t₅₀ and lag time) of hCT as determined
by ThioT fluorescence, turbidity and HDX-MS measurements

| Measurement | Parameter (min) | hCT |
|---|---|---|
| ThioT | t₅₀ | 236 ± 8 |
| fluorescence^{a} | lag time | 172 ± 15 |
| Turbidity^{a} | t₅₀ | 257 ± 15 |
| | lag time | 198 ± 20 |
| HDX-MS^{b} | t₅₀ | 165 ± 7 |
| (Intact level) | lag time | 104 ± 7 |

^{a}n = 6,
^{b}n = 3,
mean ± SD

TABLE 2
Fibrillation kinetic parameters, t₅₀ and lag time, of phosphorylated
hCT's as determined by ThioT fluorescence measurements

| Measurement | Parameter | pSer-5 hCT^{a} | pThr-13 hCT | pThr-21 hCT^{a} |
|---|---|---|---|---|
| ThioT | t₅₀ | 83 ± 8 | NA | 923 ± 89 |
| fluorescence | lag time | 58 ± 8 | NA | 568 ± 95 |

NA: not applicable,
^{a}n = 6,
mean ± SD

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected. It is intended that the scope of the present methods and apparatuses be defined by the following claims. However, it must be understood that this disclosure may be practiced otherwise than is specifically explained and illustrated without departing from its spirit or scope.

### REFERENCES

1. Moorthy, B. S.; Ghomi, H. T.; Lill, M. A.; Topp, E. M., Structural transitions and interactions in the early stages of human glucagon amyloid fibrillation. Biophys J 2015, 108 (4), 937-948.
2. Zhang, J.; Mao, X.; Xu, W., Fibril nucleation kinetics of a pharmaceutical peptide: the role of conformation stability, formulation factors, and temperature effect. Molecular pharmaceutics 2018, 15 (12), 5591-5601.
3. Biancalana, M.; Makabe, K.; Koide, A.; Koide, S., Molecular mechanism of thioflavin-T binding to the surface of beta-rich peptide self-assemblies. J Mol Biol 2009, 385 (4), 1052-63.
4. Kammari, R.; Topp, E. M., Solid-State Hydrogen-Deuterium Exchange Mass Spectrometry (ssHDX-MS) of Lyophilized Poly-d,l-Alanine. Mol Pharm 2019, 16 (7), 2935-2946.
5. Kheterpal, I.; Wetzel, R., Hydrogen/deuterium exchange mass spectrometry a window into amyloid structure. Accounts of chemical research 2006, 39 (9), 584-593.
6. Gade Malmos, K.; Blancas-Mejia, L. M.; Weber, B.; Buchner, J.; Ramirez-Alvarado, M.; Naiki, H.; Otzen, D., ThT 101: a primer on the use of thioflavin T to investigate amyloid formation. Amyloid 2017, 24 (1), 1-16.
7. Kamgar-Parsi, K.; Hong, L.; Naito, A.; Brooks, C. L., 3rd; Ramamoorthy, A., Growth-incompetent monomers of human calcitonin lead to a noncanonical direct relationship between peptide concentration and aggregation lag time. J Biol Chem 2017, 292 (36), 14963-14976.

1. A modified peptide, or a pharmaceutically acceptable salt thereof, wherein said modified peptide has no or substantially reduced fibrillation whereby said modified peptide produces a stable aqueous solution, due to said modification.

2. The modified peptide according to claim 1, wherein said modification is phosphorylation of one or more side chains of amino acid residues of said peptide.

3. The modified peptide according to claim 2, wherein said modified peptide comprises the amino acid sequence of SEQ ID NO: 1 (human calcitonin), or a substantial fragment thereof, modified such that said modified peptide has no or substantially reduced fibrillation whereby said modified peptide produces a stable aqueous solution.

4. The modified peptide according to claim 2, wherein said modified peptide comprises the amino acid sequence of SEQ ID NO: 2 (human amylin peptide), or a substantial fragment thereof, modified such that said modified peptide has no or substantially reduced fibrillation whereby said modified peptide produces a stable aqueous solution.

5. The modified peptide according to claim 3, wherein said modification is carried out on the side chain of Ser5, Thr6, Thr11, Tyr12, Thr13, His20, Thr21, Thr25, or a combination thereof.

6. The modified peptide according to claim 4, wherein said modification is carried out on the side chain of Thr4, Thr6, Thr9, His18, Ser19, Ser20, Ser28, Ser29, Thr30, Ser34, Thr36, Tyr37, or a combination thereof.

7. A pharmaceutical composition comprising:
a. a phosphorylated peptide or a pharmaceutically acceptable salt thereof, wherein said modified peptide has no or substantially reduced fibrillation whereby said phosphorylated peptide produces a stable aqueous solution, due to said phosphorylation; and
b. one or more pharmaceutically acceptable diluents, excipients or carriers.

8. The pharmaceutical composition of claim 7, wherein said modified peptide comprises the amino acid sequence of SEQ ID NO: 1 (human calcitonin), or a substantial fragment thereof, modified such that said modified peptide has no or substantially reduced fibrillation whereby said modified peptide produces a stable aqueous solution.

9. The pharmaceutical composition of claim 7, wherein said modified peptide comprises the amino acid sequence of SEQ ID NO: 2 (human amylin peptide), or a substantial fragment thereof, modified such that said modified peptide has no or substantially reduced fibrillation whereby said modified peptide produces a stable aqueous solution.

10. The pharmaceutical composition of claim 8, wherein said modification is carried out on the side chain of Ser5, Thr6, Thr11, Tyr12, Thr13, His20, Thr21, Thr25, or a combination thereof.

11. The pharmaceutical composition of claim 9, wherein said modification is carried out on the side chain of Thr4, Thr6, Thr9, His18, Ser19, Ser20, Ser28, Ser29, Thr30, Ser34, Thr36, Tyr37, or a combination thereof.

12. (canceled)

13. (canceled)

14. (canceled)

15. (canceled)

16. (canceled)

17. A method of administering calcitonin to a patient in need thereof, which method comprises administering to the patient a therapeutically effective amount of the pharmaceutical composition of claim 8.

18. The method of claim 17, wherein the side chain of Ser5, Thr6, Thr11, Tyr12, Thr13, His20, Thr21, Thr25, or a combination thereof in the amino acid sequence of SEQ ID NO:1 is modified.

19. A method of administering amylin to a patient in need thereof, which method comprises administering to the patient a therapeutically effective amount of the pharmaceutical composition of claim 9.

20. The method of claim 19, wherein the side chain of Thr4, Thr6, Thr9, His18, Ser19, Ser20, Ser28, Ser29, Thr30, Ser34, Thr36, Tyr37, or a combination thereof in the amino acid sequence of SEQ ID NO: 2 is modified.
